(19) United States
(12) Patent Application Publication
(10) Pub. No.: US 2019/0106360 A1
(43) Pub. Date: Apr. 11, 2019

(54) OSTEOSYNTHESIS BODY OF ZIRCONIUM DIOXIDE CERAMIC

(71) Applicants: Michael GAHLERT, Munchen (DE); Stefan GAHLERT, Stuttgart (DE)

(72) Inventors: Michael GAHLERT, Munchen (DE); Stefan GAHLERT, Stuttgart (DE)

(21) Appl. No.: 16/211,424

(22) Filed: Dec. 6, 2018

Related U.S. Application Data
(63) Continuation of application No. PCT/EP2017/063548, filed on Jun. 2, 2017.

(30) Foreign Application Priority Data
Jun. 9, 2016 (DE) 10 2016 110 512.0

(51) Int. Cl. C04B 35/486; C04B 35/645; A61B 17/80; A61C 8/00; A61L 31/02

(52) U.S. Cl. CPC C04B 35/486; A61B 2017/0088; A61B 17/8071; A61C 8/0012; A61C 8/0022; A61L 31/026; C04B 2235/3246; C04B 2235/3244; C04B 2235/3217; C04B 2235/6567; C04B 2235/5409; C04B 2235/6026; C04B 2235/6027; C04B 2235/612; C04B 2235/661; C04B 2235/765; C04B 2235/96; C04B 35/6455

(57) ABSTRACT

An osteosynthesis body consisting of a zirconium dioxide ceramic in the form of a screw or a shaped body for osteosynthesis such as a panel or a strip in a planar or three-dimensionally curved form which is provided in at least some sections with holes through which screws can be passed. This is preferably produced from TZP powder by means of HIP and a subsequent post-processing step in order to achieve the highest possible mechanical resilience from the finest possible powders, with an average specific surface area of, for example, 30 to 50 m2/g.

### CROSS-REFERENCES TO RELATED APPLICATIONS

This application is a continuation of international patent application PCT/EP2017/063548, filed on Jun. 2, 2017 designating the U.S., which international patent application has been published in German language and claims priority from German patent application 10 2016 110 512.0, filed on Jun. 9, 2016. The entire content of these priority applications is incorporated herein by reference.

### BACKGROUND

The invention relates to osteosynthesis bodies. In accident surgery since the 60s for the open reduction and internal fixation of bone fractures osteosynthesis bodies made of metal have been used. Initially to this end almost exclusively stainless steel was used. However, it appeared that osteosynthesis steel is not completely free from corrosion and may lead to the release of metal intolerances and allergies. As a problem in particular the partially toxic effects of chromium, nickel and iron have turned out. Therefore, for decades bodies of titanium have been used for osteosynthesis which due to surface passivation forming titanium dioxide are widely regarded as bio-inert.

Although a use of titanium leads to significantly lower rates of infection and allergic reactions, still sporadically there occur intolerance reactions.

Further from CN 2862995 Y an internal fixation from a composite material of hydroxyapatite and zirconium dioxide is known. However, this is a composite material, namely a combination of hydroxyapatite and zirconium dioxide. The detailed design and the technical implementation of this composite material cannot be derived from this document. In medicine, zirconium dioxide already has been used as a material for joint bodies in artificial hip joints. Also in dentistry zirconium dioxide has established itself as a material for endodontic posts, as a scaffold for all-ceramic crowns or bridges. Since the early 2000s zirconium dioxide has also been used as a material for fully ceramic implants (see e.g. WO 03/045268 A1).

However, a transfer of these applications to an application of zirconium dioxide for osteosynthesis is not obvious, since this is excluded by the high demands with respect to the mechanical stability and strength of osteosynthesis bodies.

### SUMMARY

In view of this it is a first object of the invention to disclose an osteosynthesis body and a method for producing such, wherein a particularly good compatibility is achieved and allergic reactions are largely avoided.

In view of this it is a second object of the invention to disclose an osteosynthesis body in the form of a screw and a method for producing such consisting of a highly dense zirconium dioxide ceramic having a very high strength.

In view of this it is a third object of the invention to disclose an osteosynthesis body in the form of a screw that is particularly suited for use in dentistry for fixing bone substitute material when building up bone substitutes for the preparation of the later setting of implants.

According to a first aspect of the invention this object is solved by an osteosynthesis body made of zirconium dioxide ceramic, in the form of a screw, consisting of a highly dense zirconium dioxide ceramic having a porosity of less than 0.1%, made of tetragonal, polycrystalline zirconium dioxide (TZP) or made of zirconium dioxide mixture ceramic with Al2O3(ATZ).

The object of the invention is completely solved in this way.

Additional developments of the invention are protected in the dependent claims.

It has been found that osteosynthesis bodies of zirconium dioxide ceramic are bio-inert and exhibit good osteointegration characteristics. In addition, it has been found that in the meantime mechanically highly stable moldings and screws made of pure zirconium dioxide ceramic can be produced which have a high flexural strength and fracture toughness and a low elastic modulus, and that therefore are suitable for osteosynthesis. In particular when using TZP (tetragonal, polycrystalline zirconium dioxide), high-strength molded bodies which are suitable for osteosynthesis can be produced.

The osteosynthesis plates may be configured as “classic” osteosynthesis plates that do not effect an angular stable fixation. However, also angular stable osteosynthesis bodies can be made of zirconium dioxide ceramic, which in combination with the corresponding screws of zirconium dioxide ceramic allow for angularly stable bone fixation.

Preferably osteosynthesis bodies consisting of a highly dense zirconium dioxide ceramic of a tetragonal, polycrystalline zirconium dioxide (TZP) are used, with a porosity of less than 0.01%.

These are characterized by a particularly high strength.

Basically there are two alternatives for the application of osteosynthesis bodies of zirconium dioxide. The first alternative is an only temporary use in the fixation of fractures. Herein the good compatibility and inertness of the material is in the foreground. The respective osteosynthesis material after sufficient healing time (usually several months) is removed again, as is also the case with osteosynthesis bodies of steel or titanium. In such a case, a smooth surface is used, i.e. a surface as produced by the production process (usually sintering), without requiring a special surface roughening treatment. In addition, a smoothing may be provided, such as by a final grinding treatment.

However, if the osteosynthesis bodies shall remain permanently within the body, which is made possible by the inertness of the material, then also a particularly good osteointegration is important.

It has been found that excellent osteointegration can be achieved in particular by means of a micro-rough surface. Such a micro-rough surface can in particular be reached, in that the outer surface is surface treated by means of a subtractive method, in particular is etched and/or blasted, in particular sand blasted. To this end in particular mild sand blasting methods with a subsequent etching treatment, for instance by means of hydrofluoric acid, are in the foreground.

When the osteosynthesis bodies remain in the body permanently, the good osteointegration characteristics in combination with the osteointegrative capacity of zirconium dioxide can be exploited.

In particular, in skeletal regions, wherein the mechanical stresses do not reach the high loads of the walking apparatus, that are present in the legs and feet, the somewhat lower mechanical strength of zirconium dioxide, in particular with regard to the tensile strength and flexural strength when compared to metallic materials, such as titanium or stainless steel, can be accepted.

In particular a utilization of osteosynthesis bodies of zirconium dioxide in the head region, but also partially in hand, arm or shoulder area, is in the foreground. Of particular interest is an application in the head area, such as major fractures, guns shot wounds, war wounds, etc.

The object of the invention is further achieved by a method of producing an osteosynthesis body from a zirconium dioxide ceramic made of a tetragonal, polycrystalline zirconium dioxide (TZP), wherein an osteosynthesis body is formed and sintered form a TZP powder.

If for instance with 3 weight-% Y2O3 partially stabilized TZP is used, then a bending strength of 1200 MPa can be achieved.

In addition, it is also conceivable to use a zirconium dioxide mixture ceramic, which is reinforced with alumina (ATZ). This material has partially a higher bending strength and possibly also an increased fracture toughness.

This is of particular interest for the plate osteosynthesis.

Very high strength can be obtained (flexural strength up to 1300 MPa at TZP), when the shaping and sintering is done simultaneously by hot-isostatic pressing (HIP), followed by a mechanical processing to produce the desired shape, which usually is carried out by grinding, milling or drilling with diamond tools.

Since herein, however, in general diamond tools must be used and by means of HIP only rough preforms, such as in the form of plates or cylinders, may be produced by HIP, this is a very time-consuming and expensive procedure.

Alternatively, therefore also a shaping may be effected by conventional powder technological methods, such as slip casting, centrifugal casting, uniaxial pressing, isostatic pressing or electrophoretic deposition (EPD).

Preferably, thereafter initially a pre-sintering to form a green body is done that subsequently in the green state is mechanically machined, in particular by milling, cutting, grinding or drilling, and which is finally sintered to its final state.

This allows for an easy shaping of the osteosynthesis body by means of mechanical machining in the green state. In this way, also an individual adaptation to individual patients is possible.

If the shrinkage measurements are taken into account during the sintering process sufficiently precisely, which can be done for example by CAD/CIM shaping by means of automatically controlled milling tools, then after sintering no or only minimal post-processing is usually required.

According to a further variant of the invention, it is possible to mix the powder with an organic binder to form a printable paste, applied by means of a 3D printer, and subsequent sintering.

During sintering the organic components evaporate, and a highly dense body of low porosity is generated. By means of 3D printing various forms can be produced in a particularly simple way. In this way also a patient-specific adaptation is possible.

According to another embodiment of the invention, microscale powder having an average specific surface area in the range of 5 to 100 m2/g is used. In particular, a larger specific surface area in the range of 30 to 50 m2/g, or even more, is particularly preferable, because thereby particularly good sintering properties result, and highly dense bodies of low porosity can be produced. Also lower sintering temperatures result.

According to a further embodiment of the invention a nanoscale powder having an average specific surface area of more than 100 m2/g, preferably of more than 200 m2/g is used. Nanoscale TZP zirconium dioxide powders are meanwhile commercially available. For producing, chemical vapor synthesis (CVS) is typically used.

By using such a powder with such a large specific surface area an even higher strength can be generated, which is especially advantageous for the use as osteosynthesis body.

In particular, a mixture of microscale and nanoscale powder is particularly advantageous.

The sintering temperature preferably is in the range of 860° C. to 1800° C., more preferred in the range of 850° C. to 1550° C.

The sintering temperature is in particular dependent from the surface characteristics of the powder, and in particular from the average specific surface area, i.e. the finer the powder is, the lower is usually the sintering temperature.

When using a nanoscale zirconium dioxide powder, therefor the sintering preferably is in the range of 850° C. to 1050° C., preferably in the range of 900° C. to 1000° C.

As far as a pre-sintering to a green body is performed, which is sufficiently stable for a mechanical treatment in the green state, then this is done usually at a pre-sintering temperature which is 100 K to 500 K below the sintering temperature.

It will be understood that the afore-mentioned features and the features to be explained hereinafter cannot only be used in the respectively given combination, but also in different combinations or independently, without departing from the scope of the invention.

### BRIEF DESCRIPTION OF THE DRAWINGS

Further features and advantages of the invention can be taken from the subsequent description of preferred embodiments with reference to the drawings. In the drawings show:

FIG. 1 an exemplary embodiment of an osteosynthesis plate according to the invention consisting of TZP; and

FIG. 2 a perspective view of a bone screw according to the invention, made of zirconium dioxide, intended for osteosynthesis, which is provided with a self-cutting thread.

### PREFERRED EMBODIMENTS

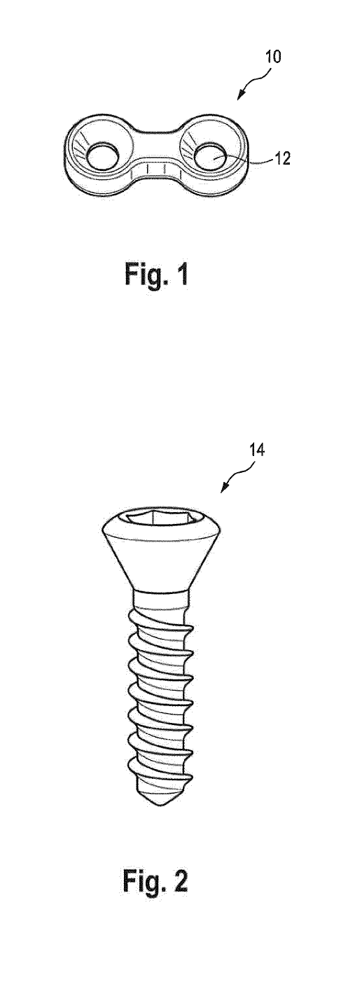

In FIG. 1 an osteosynthesis plate 10 is shown which is configured as an angularly stable plate, such as for instance be used in mandibular fracture treatment. The osteosynthesis plate 10 has two cone-shaped recessed screw holes 12 for receiving osteosynthesis screws 14 according to FIG. 2.

For preparing the osteosynthesis plate 10 initially a commercial available TCP zirconium dioxide powder (stabilized with 3 weight-% Y2O3) with an average specific surface area of about 50 m2/g is isostatically pressed (pressure about 1000 to 3000 bars, for 1 to 100 seconds), is thereafter sintered at about 900° C., for about 30 minutes.

The green body obtained in this way is machined to the desired final shape by means of an automatically controlled milling device, wherein the degree of shrinkage is taken into account for the subsequent sintering process. Thereafter, a final sintering is carried out at about 1350° C. over a period of about 30 to 60 minutes.

A particularly cost-effective manufacture is obtained, in case the pre-sintered green body is prepared by slip casting.

By means of an automatically controlled high-speed milling cutter or grinder with a diamond tool, the osteosynthesis plate 10 is prepared therefrom.

To prepare the osteosynthesis screws 14 for the purpose of very high strength, TCP powder according to the above example is pressed by HIP (e.g. 1000 to 3000 bars, 1200 to 1300° C., 2-30 minutes). From this the screw 14 is prepared by milling or grinding using diamond tools.

It is basically possible to use osteosynthesis screws 14 as shown in FIG. 2, which are provided with a self-cutting thread.

Due to the thereby occurring higher surface pressure, partially non-self-cutting screws are preferred which for example have a fine thread and slightly taper conically (not shown). Although this means higher demands on the operator, as a thread cutting is required, however it means a higher load safety.

If the osteosynthesis system consisting of the osteosynthesis plate 10 and the respective screws is intended only for temporary fixation, then the surface of the osteosynthesis plate 10 must not be additionally surface-treated after sintering. Possibly a machine smoothing by a grinding treatment is performed.

By contrast, in case of permanent implantation the surfaces of the osteosynthesis body (that is plates 10 and screws 14) undergo a mild sandblast treatment, followed by an etching treatment using hydrofluoric acid, to effect a particularly good osteointegration.

What is claimed is:

1. An osteosynthesis body made of zirconium dioxide ceramic, in the form of a screw, consisting of a highly dense zirconium dioxide ceramic having a porosity of less than 0.01%, made of tetragonal, polycrystalline zirconium dioxide (TZP) or made of zirconium dioxide mixture ceramic with Al₂O₃ (ATZ), wherein an outer surface of the screw is etched or blasted.

2. The osteosynthesis body of claim 1, in the form of a non self-tapping screw.

3. The osteosynthesis body of claim 1, in the form of a self-tapping screw.

4. The osteosynthesis body of claim 1, wherein said osteosynthesis body is configured for fixing bone substitute material in bone build-up for setting dental implants.

5. The osteosynthesis body of claim 1, wherein the outer surface is sand blasted and etched.

6. An osteosynthesis body made of zirconium dioxide ceramic, in the form of a screw, consisting of a highly dense zirconium dioxide ceramic having a porosity of less than 0.1%, made of tetragonal, polycrystalline zirconium dioxide (TZP) or made of zirconium dioxide mixture ceramic with Al₂O₃ (ATZ).

7. The osteosynthesis body of claim 6 having a porosity of less than 0.01%.

8. The osteosynthesis body of claim 6, wherein the outer surface is roughened.

9. The osteosynthesis body of claim 6, wherein the outer surface is etched.

10. The osteosynthesis body of claim 6, wherein the outer surface is sand blasted.

11. The osteosynthesis body of claim 6, wherein said osteosynthesis body is configured for fixing bone substitute material in bone build-up for setting dental implants.

12. A method for producing an osteosynthesis body of a zirconium dioxide ceramic, wherein from a powder of tetragonal, polycrystalline zirconium dioxide (TZP), or of a zirconium dioxide mixture ceramic powder (ATZ) an osteosynthesis body is formed and sintered.

13. The method of claim 11, wherein the shaping is performed by a method selected from the group consisting of HIP, slip casting, centrifugal casting, uniaxial pressing, isostatic pressing, and electrophoretic deposition (EPD).

14. The method of claim 11, wherein initially a preform is formed from powder, is pre-sintered to a green body, is subsequently in the green state mechanically machined, and is finally sintered to its final state.

15. The method of claim 11, wherein the powder is mixed with a binder to a printable paste, is applied by means of a 3D printer and is sintered thereafter.

16. The method of claim 11, wherein a microscale powder having an average specific surface area in the range of 5 to 100 m g²/g is used.

17. The method of claim 11, wherein a nanoscale powder having a specific surface area of more than 100 m²/g, preferably of more than 200 m²/g is used.

18. The method of claim 11, wherein microscale and nanoscale powders are mixed.

19. The method of claim 11, wherein the sintering is performed in the 850° C. to 1350° C.

20. The method of claim 11, wherein a pre-sintering to a green body is performed, the green body is mechanically machined thereafter, and is sintered thereafter to its final state.
